(19) United States
(12) Patent Application Publication
(10) Pub. No.: US 2008/0014547 A1
(43) Pub. Date: Jan. 17, 2008

(54) Personal visual scope

(76) Inventor: Antonio Carabillo, Closter, NJ (US)

Correspondence Address: WARD & OLIVO, SUITE 300, 382 SPRINGFIELD AVENUE, SUMMIT, NJ, 07901, US

(21) Appl. No.: 11/799,872

(22) Filed: May 2, 2007

Related U.S. Application Data
(60) Provisional application No. 60/796,639, filed on May 2, 2006.

(51) Int. Cl. A61B 1/247

(52) U.S. Cl. 433/31

(57) ABSTRACT

This invention relates to an instrument having a handle and a mirror hingedly attached to an end of the handle. The instrument may be adapted to a number of applications. The mirror may be fitted with a plurality of LED lights that may surround the periphery of the mirror. The LEDs may also be positioned in the handle in close proximity to the mirror. A power source is contained within the handle and is connected to the LED.

### RELATED SUBJECT MATTER

This application claims the benefit of, under 35 U.S.C. § 119(e), U.S. Provisional Application No. 60/796,639 filed May 2, 2006.

### BACKGROUND OF THE INVENTION

The present invention relates to a mirrored instrument. An example of such an instrument is a Butler Plakfinder commonly used in dental applications. Such instruments have a wide range of uses. An important benefit of indirect vision is to allow a dental health care provider by reflecting light onto desired surfaces

Indirect vision is needed in certain locations of the mouth where visibility is difficult or impossible. The posterior surfaces of the anterior maxillary teeth are a notable area where Butler Plakfinder are often employed. Other areas of the mouth can be viewed more readily with the Butler Plakfinder, even though it would be possible to see them if the dentist or dental hygienist adjusted their body into a poor position.

There are other areas of the mouth where lighting is difficult, even with the over head lights used regularly in dental offices. In these instances, the Butler Plakfinder is used to reflect light onto those surfaces. This is especially useful if the mirror is simultaneously being used for indirect vision of an obscure area.

Where access is difficult, such instruments are limited in their usefulness. A fixed mirror is not suitable for many applications. Also, such instruments are limited in their useful by the amount ambient light available.

### SUMMARY OF THE INVENTION

Accordingly, it is an object of the invention to provide an instrument that provides an adjustable mirror for enhanced flexibility, durability and precision.

It is also an object of the invention to provide an instrument which provides light and visibility to poorly lit areas through a number of LED lights positioned in the vicinity of the mirror.

It is a further object of the invention to provide an instrument which eliminates the need for reflecting indirect light.

These and other objectives are achieved through the provision of an instrument having a handle and a mirror hingedly attached to an end of the handle. The instrument may be adapted to a number of applications. One example is as a dental instrument for professional use or alternatively to be attached to a toothbrush for personal use. Another application is for personal or general use, such as on a key chain. The mirror may be fitted with a plurality of LED lights that may surround the periphery of the mirror. The LEDs may also be positioned in the handle in close proximity to the mirror. A power source is contained within the handle and is connected to the LED.

### BRIEF DESCRIPTION OF THE DRAWINGS

In the drawings, which constitute a part of this specification, exemplary embodiment exhibiting various objectives and features hereof are set forth, specifically:

FIG. 1 is a diagrammatic view of the visual scope in accordance with the present invention.

### DETAILED DESCRIPTION OF THE INVENTION

As indicated above, a detailed illustrative embodiment of the present invention is disclosed herein. However, elements in accordance with the present invention may be embodied in a wide variety of forms some of which may be quite different from those of the disclosed embodiment. Consequently, the specific structural and functional details disclosed herein are merely representative; yet in that regard, they are deemed to afford the best embodiment for purposes of disclosure and to provide a basis for the claims herein which define the scope of the present invention.

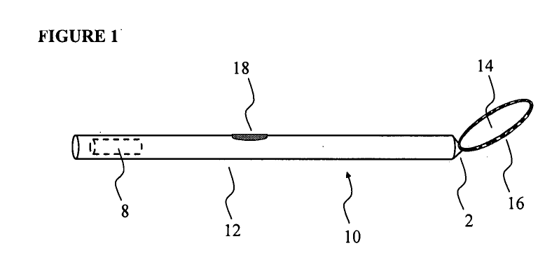

The instrument 10 illustrated in FIG. 1, uses a modified version of the Butler Plakfinder. The instrument 10 includes an elongated handle 12. The handle 12 may be of a cylindrical or rectangular armature. The handle 12 may also be solid, flexible, or telescoping.

The handle 12 is fitted with a mirror 14 at one end. The mirror 14 helps to reflect light in dark areas, and also provides visibility to hidden areas. The mirror 14 may be hingedly attached. It may also be able to swivel in any direction. Miniature LEDs 16 may surround or outline the mirror 14. At least one LED may be preferably located in the handle 12.

Alternatively, the LED may be positioned only on the handle and not surrounding the mirror. The LED 16 in the handle can be directed forward in the direction of the hinged mirror 14. In another embodiment, the LED may be pitched inward or straight up for optimal lighting and visibility.

A switch 18 to operate the LEDs 18 may be located along the handle. A power source, which may be located within the handle, may be a rechargeable or mini-high power alkaline battery.

In another embodiment, the instrument may be incorporated onto the bottom of a toothbrush. The instrument may use a mirror with a push-button switch attached within. For example, the mirror when nominally closed is folded against the handle and when opened, activates the LEDs.

Another embodiment of the instrument may be adapted to a keychain. The mirror would fold up, using a hinge or swivel, into the housing which contains the battery and switch. The switch would default to the off settings. The alternate side of the housing or mirror may contain a magnifying or telescoping lens which also illuminates when opened. When the mirror and lens are folded closed or into housing, the system or design may be used as a flashlight.

While the preferred embodiment of the invention has been illustrated and described, it will be appreciated that various changes can be made therein without departing from the spirit and scope of the invention. Furthermore, it will be apparent that while the disclosed embodiment comprises specific elements and configurations, any of a variety of structure might well be utilized. Accordingly, the scope hereof is deemed to be as set forth in the claims below.

What is claimed is:

1. A instrument comprising:
a handle;
a mirror positioned at an end of said handle;
at least one LED positioned in the vicinity of said mirror;
a hinge connecting said handle to said mirror;
a power source contained within said handle; and
a switch to operate the LED.
